(19) United States
(12) Patent Application Publication
(10) Pub. No.: US 2026/0354743 A1
(43) Pub. Date: Nov. 12, 2026

(54) SWALLOWING MEASUREMENT

(71) Applicant: Hoffmann-La Roche Inc., Little Falls, NJ (US)

(72) Inventors: Roman Peter KUSTER, Basel (CH); Thanneer Malai PERUMAL, Oberwil, Basel-Landschaft (CH)

(21) Appl. No.: 19/118,338

(22) PCT Filed: Oct. 6, 2023
(86) PCT No.: PCT/EP2023/077723

§ 371 Date: Apr. 3, 2025

(30) Foreign Application Priority Data
Oct. 7, 2022 (EP) 22200280.0

(51) Int. Cl. A61B 5/00; G16H 50/20

(52) U.S. Cl. CPC A61B 5/4205; A61B 5/0002; A61B 5/6898; A61B 5/7282; A61B 5/742; A61B 5/7475; G16H 50/20

(57) ABSTRACT

A diagnostic device configured to count a number of swallows of a user, the device comprising: at least one processor; a user interface; one or more sensors associated with the device; and a memory storing computer-readable instructions that, when executed by the at least one processor, cause the diagnostic device to: prompt, via the user interface, the user to provide a user input via the one or more sensors associated with the device each time the user is at a predetermined point during a swallowing action; receive a plurality of inputs via the one or more sensors, each user input corresponding to a respective time at which the user is at a predetermined point during the swallowing action; generate, in response to receiving each user input, a timestamp associated with the respective user input; apply a counting model to data comprising the plurality of generated timestamps, wherein the counting model is configured to calculate a number of swallows of a user by counting the total number of timestamps in the data comprising the plurality of generated timestamps; and outputting the calculated number of swallows.

### TECHNICAL FIELD OF THE INVENTION

The present invention relates to diagnostic devices and computer-implemented methods of determining how many times a subject has swallowed.

### BACKGROUND TO THE INVENTION

Spinal Muscular Atrophy (SMA) is associated with bulbar weakness. People living with SMA (PlwSMA) are often affected by swallowing difficulties (dysphagia). The degree of dysphagia ranges from minor issues with swallowing certain food or liquid types (e.g. very hard or dry food, very thin liquids), to the need for more swallows for a bolus, up to the complete inability to swallow with full dependence on tube feeding {van der Heul et al. 2019, PMID 31476167}. However, tube feeding does not eliminate swallowing completely; many tube fed patients have regular oral intake for pleasure, and they still have to deal with their saliva (approximately 1 litre or 300 swallows a day).

Even though the progress of dysphagia is thought to be slow, it is not rare that serious deteriorations are reported to clinicians as being sudden, probably as a result of coping strategies and missing tools to track longitudinal progression. PlwSMA with dysphagia might have difficulties to get enough nutrients to maintain their body weight and they are at risk of (silent) penetration and aspiration, where food or liquid enters the airways. Especially when silent (not noticed by the person) or in combination with a weak cough (insufficient airway clearance), penetration and aspiration could lead to life-threatening airway infections {McGrattan et al. 2021, PMID 33822657}.

PlwSMA reporting issues with swallowing might undergo a video-fluoroscopic swallowing study (VFSS) or, in an effort to limit the radiation exposure, a fibre-optic endoscopic evaluation of swallowing {Audag et al. 2019, PMID 30728931; Nacci et al. 2008, PMID 18939710}. The primary aim of VFSS is to derive safe food and drink recommendations for the individual, among others, with the help of global clinical scales such as the ‘Penetration Aspiration Scale’ or the ‘Dysphagia Outcome Severity Scale’ to grade the severity of the observed penetration and/or aspiration {Rosenbek et al. 1996, PMID 8721066; O'Neil et al. 1999, PMID 10341109}. These recommendations often include avoiding dry and hard foods and/or avoiding thin liquids (which is why the swallowing of saliva bears a great risk). However, the output of VFSS is subjective and depends on the examiner's experience, as outlined in the SMALL talk of Katlyn McGrattan. Despite the prevalence of dysphagia in PlwSMA, it is worth noting that in clinical practice, VFSS is rare and conducted only on serious suspicion (e.g. when serious swallowing issues were already reported). VFSS is not a tool to track swallowing difficulties longitudinally. Other patient reported outcomes, such as the ‘Neuromuscular Disease Swallowing Status Scale’ or the ‘Sydney Swallowing Questionnaire’, exist but are rarely used systematically in PlwSMA {Audag et al. 2019, PMID 30728931}.

It is therefore desirable to provide a method of collecting swallowing data from patients which does not give rise to the issues outlined above.

### SUMMARY OF THE INVENTION

The present invention, at a high-level, provides a device and computer-implemented method of determining a number of times a subject has swallowed, for example within a given amount of time. More specifically, a user is prompted to provide an input to the device each time they reach a predetermined point in an swallowing cycle. From a series of inputs, the device is then able to calculate a number of times that the user has swallowed.

Accordingly, a first aspect of the present invention provides a diagnostic device configured to measure a number of times a subject swallows, the device comprising: at least one processor; one or more sensors associated with the device; a user interface; and a memory storing computer-readable instructions that, when executed by the at least one processor, cause the diagnostic device to: prompt, via the user interface, the user to provide a user input, via the one or more sensors associated with the device, each time the user is at a predetermined point during a swallowing action; receive a plurality of user inputs via the one or more sensors, each user input corresponding to a respective time at which the user is at the predetermined point during a swallowing action; generate, in response to receiving each user input, a timestamp associated with the respective user input; apply a counting model to data comprising the plurality of generated time stamps, wherein the counting model is configured to calculate a number of swallows of a subject by counting the total number of timestamps in the data comprising the plurality of generated timestamps; and output the calculated number of swallows.

The combination of the steps of: prompting the user; receiving the plurality of user inputs; generating the timestamps; applying the counting model; and, outputting the calculated number of swallows may correspond to conducting a “swallow test”. That is, the computer-readable instructions, when executed by the at least one processor, may cause the device to conduct a swallow test, which may include the above-mentioned steps.

It is known that a number of swallows, e. g. in a given amount of time may correlate with validated clinical tools for assessing dysphagia such as EAT-10 (eating assessment tool 10) or SSQ (Sydney Swallow Questionnaire), both of which clinical tools can also be useful in making clinical assessments. By measuring an amount of swallowing using a diagnostic device according to the first aspect of the present invention, it may be possible to track, effectively, the progress of various muscular disabilities such as SMA in a subject by active testing of the subject. Use of diagnostic devices according to the first aspect of the invention may be particularly effective for assessment of e.g. symptom severity (i.e. difficulty in swallowing), and progression of bulbar muscular disabilities such as SMA by active testing of the subject. As is described in detail later in this application, the diagnostic device according to the first aspect of the present invention may use the calculated number of swallows to indicate and/or track the presence or progression of a muscular disability, such as SMA, in a subject or user.

Implementations of the diagnostic device may be used to assess swallowing of food and/or drink. Accordingly, the computer-readable instructions, when executed by the device, may further cause the diagnostic device to prompt the user, via the user interface, to consume a piece of food. Alternatively, or additionally, the computer-readable instructions may further cause the diagnostic device to prompt the user, via the user interface, to consume a drink. The predetermined point in the swallowing action may be the beginning or end of the swallowing action. This allows a more reliable swallowing count to be obtained, because a user is more easily able to identify the point at which they begin or end a swallowing action.

The computer-readable instructions, when executed by the device, may further cause the diagnostic device to prompt the user, via the user interface, to take a single bite from a piece of food, or a single sip from a drink. The computer-readable instructions, when executed by the device, may further cause the diagnostic device to prompt the user, via the user interface, to swallow as many times as needed to clear the mouth of the food or drink. Thus, the user may be prompted to take a single bite of food, or a single sip of a drink, and to provide a user input each time they swallow, for as many swallows as it takes to clear the mouth. The computer-readable instructions, when executed by the device, may cause the diagnostic device to receive as many user inputs as required, and/or to receive user inputs for a required period of time, corresponding to the swallows required for the user to clear the mouth. There may be no limit on the number of inputs which may be received or on the period of time over which user inputs may be received.

The computer-readable instructions, when executed by the at least one processor, may cause the diagnostic device to conduct a plurality of swallow tests. In each swallow test, the user may be prompted to consume, or take a single bite or sip of, a piece of food or drink. In the plurality of swallow tests, there may be at least one swallow test in which the user is prompted to consume, or take a single bite of, a piece of food (a “food swallow test”), and at least one swallow test in which the user is prompted to drink, or take a single sip of, a drink (a “drink swallow test”). In the plurality of swallow tests there may be a plurality, for example at least three, food swallow tests. In the plurality of swallow tests there may be a plurality, for example at least three, drink swallow tests. The computer-readable instructions, when executed by the at least one processor, may cause the diagnostic device to prompt the user, between each food swallow test, to consume one or more sips of a drink. The computer-readable instructions, when executed by the at least one processor, may conduct one or more food swallow tests and one or more drink swallow tests in alternating order.

The piece of food or drink may be a specified piece of food or drink, for example a banana, or water. The computer-readable instructions, when executed by the at least one processor, may cause the diagnostic device to display, via the user interface, an indication of the specified piece of food or drink. For example, the specified piece of food or drink may be displayed an image.

In preferred implementations, the device is or comprises a smartphone. This is advantageous because smartphones are possessed by virtually everyone nowadays. By implementing a computer-implemented process such as the one described on a smartphone, a user need not attend e.g. a hospital or other clinical setting in order for the number of swallows to be measured. Other kinds of diagnostic device may be used, e.g. a tablet, a laptop computer, a desktop computer, or the like. Alternatively, the diagnostic device may be a dedicated swallowing measuring device.

The diagnostic device preferably further comprises a display component which is configured to display the user interface. Preferably, the display component is in the form of a screen such as a touchscreen. In implementations in which the display component comprises a touchscreen, the touchscreen preferably includes the one or more sensors associated with the device. In those cases, the sensors may comprise resistive sensors, capacitive sensors, surface acoustic wave sensors, infrared grid sensors, infrared acrylic projection sensors, optical imaging sensors, piezoelectric sensors, and/or acoustic pulse recognition sensors. In most cases the one or more sensors are capacitive sensors, since these are most commonly used in smartphones. Capacitive sensors work on the basis that when a person touches the screen, its electrostatic field is distorted, which registers in a change in capacitance.

The counting model may be configured to count the number of times a subject swallows within a predetermined time range or duration. In some cases, rather than or in addition to counting the raw number of swallows, the diagnostic model may be configured to determine, derive or measure a swallowing rate. In such cases, the computer-readable instructions may, when executed by the at least one processor, be configured to cause the diagnostic device to apply (either as well as or in addition to the counting model) a swallowing rate model to the data comprising the plurality of timestamps in order to calculate a swallowing rate. The swallowing rate model may be configured to calculate a time difference between two time stamps of the plurality of timestamps, and to calculate the swallowing rate based on a reciprocal of the calculated time difference. In some cases, the earlier of the two time stamps may immediately precede the later of the two time stamps. In other words, the two time stamps may be consecutive time stamps. Alternatively, there may be n time stamps between the earlier of the two time stamps and the later of the two time stamps, and the swallowing rate model is configured to calculate the swallowing rate by multiplying the reciprocal of the time difference by (n+1). By inverting the time difference as outlined above, the swallowing rate model is able to calculate the swallowing rate in units of swallows per second. In some cases, the swallowing rate model may be further configured to multiple the inverted time difference (i.e. the reciprocal) by sixty in order to obtain the swallowing rate in units of swallows per minute. Note that a time difference between two consecutive time stamps may correspond to a swallow duration. In some examples, the swallowing rate model may be configured to calculate a mean swallow duration by summing a plurality of time differences, each time difference being the difference between two consecutive time stamps, and dividing the sum by n, where n is the number of time differences. The swallowing rate model may then calculate a mean swallowing rate by taking the inverse of the mean swallow duration.

We now discuss how the calculated number of swallows may be used to indicate a presence or a progression of a muscular disability, such as SMA. The computer-readable instructions, when executed by the at least one processor, may cause the diagnostic device to apply a clinical interpretation model to the calculated number of swallows. The clinical interpretation model may output an indication of the presence or absence of a muscular disability, such as SMA, in the user, or an indication of the progression of a muscular disability in the user. The clinical interpretation model may be configured to compare the calculated number of swallows to a predetermined value, and, based on the comparison, to output an indication of the presence or absence of the muscular disability, such as SMA. In particular, the clinical interpretation model may be configured to determine whether the calculated number of swallows is greater than a predetermined threshold, and if it is determined that the calculated number of swallows is greater than the predetermined threshold, to output an indication of the presence of a muscular disability (e.g., that the user is a PlwSMA), and/or if it is determined that the calculated number of swallows is less than or equal to the predetermined threshold, to output an indication of the absence of the muscular disability. The predetermined threshold may be less than or equal to 2, for example the predetermined threshold may be 1.

In some examples, applying the clinical interpretation model may include applying the clinical interpretation model to a plurality of calculated numbers of swallows, each of the calculated numbers of swallows calculated in a different swallow test. The clinical interpretation model may be configured to determine a minimum number of swallows in the plurality of calculated numbers of swallows. The minimum number of swallows may refer to the smallest number of swallows. In some examples, each of the calculated numbers of swallows may be calculated in a different food swallow test. In some examples, each of the calculated numbers of swallows may be calculated in a different food swallow test in which the user is prompted to take a single bite of food.

The clinical interpretation model may be configured to compare the minimum number of swallows to a predetermined value, and, based on the comparison, to output an indication of the presence or absence of the muscular disability, such as SMA. In particular, the clinical interpretation model may be configured to determine whether the minimum number of swallows is greater than a predetermined threshold, and if it is determined that the minimum number of swallows is greater than the predetermined threshold, to output an indication of the presence of a muscular disability (e.g., that the user is a PlwSMA), and/or if it is determined that the minimum number of swallows is less than or equal to the predetermined threshold, to output an indication of the absence of the muscular disability. The predetermined threshold may be less than or equal to 2, for example the predetermined threshold may be 1. PlwSMA may never, or may be very unlikely to, clear their mouth after a single bite with just a single swallow. Healthy individuals may always, or may be very likely, to clear their mouth after a single bite with just 1 swallow at least once in a series of tests. A second aspect of the present invention provides a computer-implemented method of measuring a number of swallows performed by a subject. The computer-implemented method comprises: prompting, via a user interface, the user to provide a user input, via one or more sensors associated with a device, each time the user is at a predetermined point during a swallowing action; receiving a plurality of user inputs via the one or more sensors, each user input corresponding to a respective time at which the user is at the predetermined point during a swallowing action; generating, in response to receiving each user input, a timestamp associated with the respective user input; applying a counting model to data comprising the plurality of generated time stamps, wherein the counting model is configured to calculate a number of swallows of a subject by counting the total number of timestamps in the data comprising the plurality of generated timestamps; and output the calculated number of swallows.

In preferred cases, the computer-implemented method of the second aspect of the invention is executed by a processor of a diagnostic device such as the diagnostic device of the first aspect of the invention. It will be appreciated that the optional features set out above, in respect of the first aspect of the invention, apply equally well to the second aspect of the invention except where context clearly dictates otherwise, or whether such a combination of features is clearly technically incompatible.

A third aspect of the invention provides a computer program comprising instructions which when executed by a processor of a computer (or other suitable data processing device) cause the processor to execute the computer-implemented method of the second aspect of the invention. A further aspect of the invention provides a computer-readable storage medium having stored thereon the computer program of the third aspect of the invention.

The invention includes the combination of the aspects and preferred features described except where such a combination is clearly impermissible or expressly avoided.

### BRIEF DESCRIPTION OF THE DRAWINGS

Embodiments of the present invention will now be described with reference to the accompanying drawings, in which:

FIG. 1 is a diagram of an example environment in which a diagnostic device for assessing the number of times a subject has swallowed is provided.

FIG. 2 is a flow diagram of a computer-implemented method for assessing the number of times a user has swallowed.

FIG. 3 is a flow diagram of a computer-implemented method for determining an indication of the presence or absence of a muscular disability, such as SMA.

FIG. 4 is a plot showing numbers of swallows calculated for PlwSMA and for healthy individuals.

FIG. 5 illustrates one example of a network architecture and data processing device that may be used to implement one or more illustrative aspects described herein.

### DETAILED DESCRIPTION OF THE DRAWINGS

Aspects and embodiments of the present invention will now be discussed with reference to the accompanying figures. Further aspects and embodiments will be apparent to those skilled in the art. All documents mentioned in this text are incorporated herein by reference.

In the following description of various aspects, reference is made to the accompanying drawings, which form a part hereof, and in which is shown by way of illustration various embodiments in which aspects described herein may be practiced. It is to be understood that other aspects and/or embodiments may be utilized, and structural and functional modifications may be made without departing from the scope of the described aspects and embodiments.

Aspects described herein are capable of other embodiments and of being practiced or being carried out in various ways. Also, it is to be understood that the phraseology and terminology used herein are for the purpose of description and should not be regarded as limiting. Rather, the phrases and terms used herein are to be given their broadest interpretation and meaning. The use of “including” and “comprising” and variations thereof is meant to encompass the items listed Thereafter and equivalents thereof as well as additional items and equivalents thereof. The use of the terms “mounted,” “connected,” “coupled,” “positioned,” “engaged” and similar terms, is meant to include both direct and indirect mounting, connecting, coupling, positioning and engaging.

Systems, methods and devices described herein provide a diagnostic device and computer-implemented methods for assessing, measuring, or determining a number of swallows made by a patient, for example a patient suffering from a bulbar muscular disability, such as particular SMA. In some cases, the diagnostic device may be in the form of a mobile, in particular a smartphone, on which a particular software application is installed. The software application may be configured to execute (or cause the processor of the mobile device) the corresponding computer-implemented method.

In some cases, the diagnostic obtains or receives sensor data from one or more sensors associated with the mobile device as the subject interacts with the software application using the mobile device. In some cases, the sensors may be within the mobile device. In some cases, the number of times the patient swallows is derived, calculated, or extracted from the received or obtained sensor data. In some cases, the assessment of the symptom severity and progression of a muscular disability, in particular SMA, in the subject may be determined based on the extracted sensor features.

In implementations of the present invention, the diagnostic device may prompt the subject to perform a diagnostic tasks. In some cases, the diagnostic tasks are anchored in or modelled after established methods and standardized tests. In some cases, in response to the subject performing the diagnostic task, the diagnostic obtains or receives sensor data via one or more sensors. In some cases, the sensors may be within a mobile device or wearable sensors worn by the subject. In some cases, sensor features associated with the symptoms of a muscular disability, in particular SMA, are extracted from the received or obtained sensor data. In some cases, the assessment of the symptom severity and progression of a muscular disability, in particular SMA, in the subject is determined based on the extracted features of the sensor data.

Assessments of symptom severity and progression of a muscular disability, in particular SMA, using diagnostics according to the present disclosure correlate sufficiently with the assessments based on clinical results and may thus replace clinical subject monitoring and testing. Example diagnostics according to the present disclosure may be used in an out of clinic environment, and therefore have advantages in cost, ease of subject monitoring and convenience to the subject. This facilitates frequent, in particular daily, subject monitoring and testing, resulting in a better understanding of the disease stage and provides insights about the disease that are useful to both the clinical and research community. An example diagnostic according to the present disclosure can provide earlier detection of even small changes in swallowing ability which can be indicative of the presence or progression of muscular disabilities, in particular SMA, in a subject and can therefore be used for better disease management including individualized therapy.

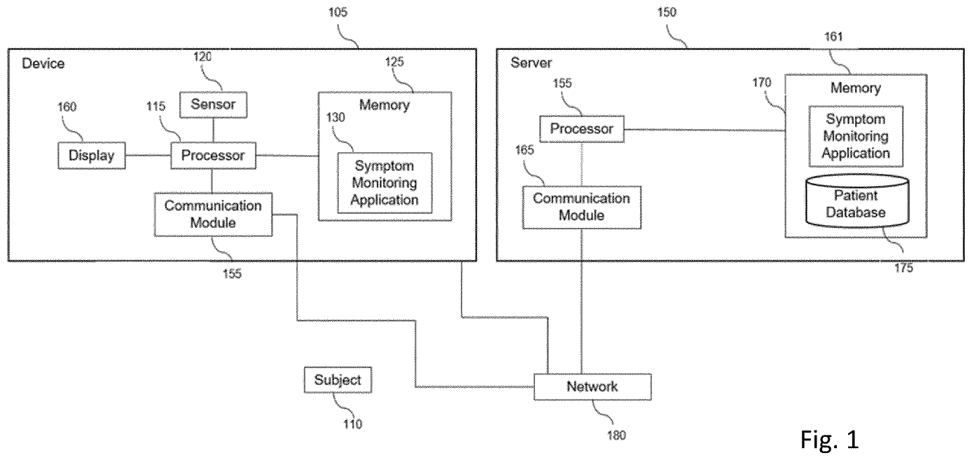

FIG. 1 is a diagram of an example environment in which a diagnostic device 105 for assessing a number of swallows made by a subject 110 having e.g. a muscular disability, in particular SMA. In some cases, the device 105 may be a smartphone, a smartwatch or other mobile computing device. The device 105 includes a display screen 160. In some cases, the display screen 160 may be a touchscreen. The device 105 includes at least one processor 115 and a memory 125 storing computer-instructions for a symptom monitoring application 130 that, when executed by the at least one processor 115, cause the device 105 to assess one or more numbers of swallows made by a subject 110 such as a patient with a muscular disability, in particular SMA, and/or to determine an indication of the presence or absence of a muscular disability such as SMA. The device 105 receives a plurality of sensor data via one or more sensors associated with the device 105. In some cases, the one or more sensors associated with the device is at least one of a sensor disposed within the device or a sensor worn by the subject and configured to communicate with the device. In FIG. 1, the sensors associated with the device 105 include a first sensor 120a that is disposed within the display screen 160 of the device 105.

The device 105 extracts, from the received first sensor data a number of swallows made by the subject 110.

The device 105 determines the number of swallows made by the subject 110 based on the extracted features. In some cases, the device 105 send the extracted features over a network 180 to a server 150. In some cases, the device 105 sends the first sensor data over the network 180 to the server 150. The server 150 includes at least one processor 155 and a memory 161 storing computer-instructions for a symptom assessment application 170 that, when executed by the server processor 155, cause the processor 155 to determine the number of swallows made by the subject 110 based on the extracted features received by the server 150 from the device 105. In some cases, the symptom assessment application 170 may determine the number of swallows made by of the subject 110 based on the extracted features of the sensor data received from the device 105 and a subject database 175 stored in the memory 160. Multiple swallow tests may be carried out, with first sensor data collected and processed in each test, such that a plurality of numbers of swallows may be determined. The symptom assessment application 170 may further determine, from the determined one or more numbers of swallows, an indication of the presence or absence of a muscular disability such as SMA and may output the indication. In some cases, the subject database 175 may include subject and/or clinical data. In some cases, the subject database 175 may include in-clinic and sensor-based measures of the number of times the subject 110 has swallowed. In some cases, the subject database 175 may be independent of the server 150. In some cases, the server 150 sends the determined one or more numbers of swallows and/or indication of the presence or absence of the muscular disability to the device 105. In some cases, the device 105 may output the number of swallows. In some cases, the device 105 may communicate information to the subject 110 based on the assessment. In some cases, the assessment of the number of swallows or the indication of the presence or absence of the muscular disability, may be communicated to a clinician that may determine individualized therapy for the subject 110 based on the assessment.

In some cases, the computer-instructions for the symptom monitoring application 130, when executed by the at least one processor 115, cause the device 105 to determine the number of swallows made by the subject 110 based on active testing of the subject 110. The device 105 prompts the subject 110 to perform one or more tasks. A number of swallows may be calculated for each task. In some cases, prompting the subject to perform the one or more diagnostic tasks includes prompting the subject 110 to eat, or take a single bite of, a piece of food, or consume, or take a single sip of, a drink, and to tap the touchscreen (or equivalent sensor) at the beginning and/or end of each swallow. The prompt may further specify the food or drink to be eaten/drunk and swallowed.

In response to the subject 110 performing each of the one or more diagnostic tasks, the diagnostic device 105 receives a plurality of sensor data via the one or more sensors associated with the device 105, the sensor data comprising a series of time stamps corresponding to the times at which a user indicates (via the sensor) when they have swallowed. The device 105 extracts, from the received sensor data for each diagnostic task the number of times that the user has swallowed, e.g. within a particular time frame. Thus a plurality of numbers of swallows may be determined. The symptoms of a muscular disability, in particular SMA in the subject 110 may include a symptom affecting the ability of the subject 110 to swallow.

Thus, the device further may further determine, from the one or more numbers of swallows, the presence or absence of a muscular disability such as SMA, for example by determining the minimum, or the smallest, number of swallows in the plurality of the numbers of swallows.

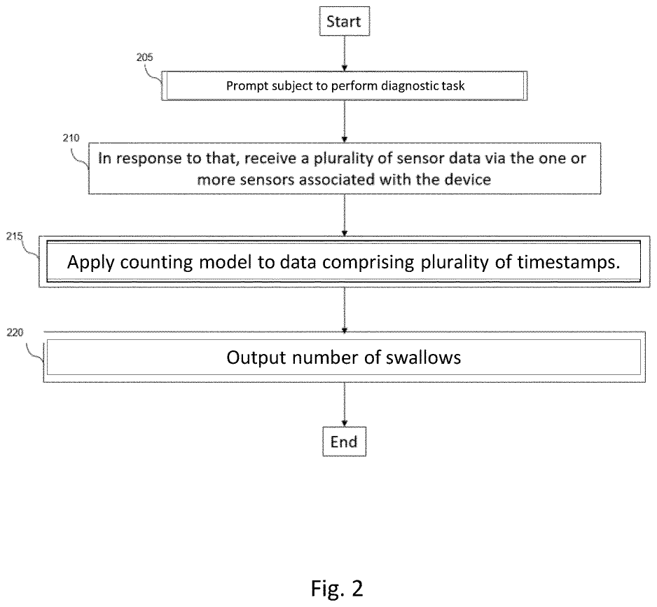

FIG. 2 illustrates an example method for assessing the number of times a user swallows of a subject in a subject based on active testing of the subject using the example device 105 of FIG. 1. While FIG. 2 is described with reference to FIG. 1, it should be noted that the method steps of FIG. 2 may be executed by other systems. The computer-implemented method includes prompting, in step 205, the subject to provide a user input on a user input interface displayed on the display 160 of the device 105 each time the subject is at a predetermined point (such as the beginning) of a swallowing action. The method includes receiving, in response to the subject performing the one or more tasks, a plurality of sensor data, via the one or more sensors (step 210), which may be in the form of capacitive sensors in the touchscreen of a display component 160.

Then, in step 215, a counting model is applied to data comprising the plurality of time stamps. The features of the counting model have been explained in detail elsewhere in this patent application, and will not be repeated here, for brevity.

In step 220, a number of swallows is output, e.g. by the processor 107 generating instructions, which when executed by the display component 160 of the device 105 cause the display component 160 to display the number of swallows. Alternatively, the calculated number of swallows may be transmitted to a server 150, as outlined elsewhere in this application.

As discussed above, assessments of symptom severity and progression of a muscular disability, in particular SMA using diagnostics according to the present disclosure correlate sufficiently with the assessments based on clinical results and may thus replace clinical subject monitoring and testing.

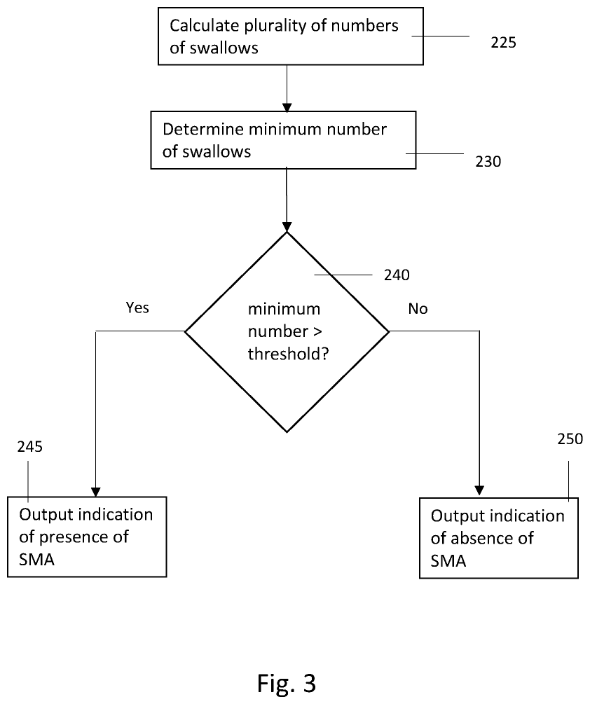

FIG. 3 illustrates an example method for determining an indication of the presence or absence of SMA in a subject based on active testing of the subject using the example device 105 of FIG. 1. While FIG. 3 is described with reference to FIG. 1, it should be noted that the method steps of FIG. 3 may be executed by other systems. The computer-implemented method includes, in step 225, calculating a plurality of numbers of swallows, each number of swallows determined according to the method described with reference to FIG. 2.

That is, each number of swallows may be calculated in a separate swallow test. The plurality of swallow tests may include only swallow tests in which the user is prompted to take a single bite of a piece of food, and may include at least 3 food swallow tests. Between each of these swallow tests, the user may have been prompted to take a sip of a drink. In step 230, the computer-implemented method includes determining the minimum number of swallows in the plurality of the number of swallows. Then, in step 240, the computer-implemented method includes determining whether the determined minimum number of swallows is greater than a predetermined threshold. If the calculated number of swallows is determined to be greater than the predetermined threshold, in step 245 the computer-implemented method includes outputting an indication of the presence of SMA. If the calculated number of swallows is determined to less than or equal to the predetermined threshold, in step 250 the computer-implemented method includes outputting an indication of the absence of SMA.

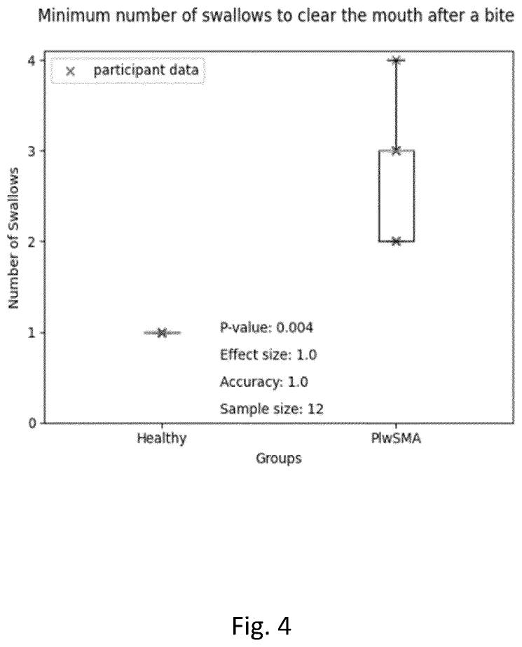

The predetermined threshold may be less than or equal to 2,for example, the predetermined threshold may be 1. That is, a minimum number of swallows of >1 may indicate that a user is a PlwSMA, and a minimum number of swallows of ≤1 may indicate that a user is not a PlwSMA. This may be explained with reference to FIG. 4. FIG. 4 is a plot showing determined minimum numbers of swallows for PlwSMA and for healthy individuals. This plot shows that all of the PlwSMA have test results of a minimum number of swallows of >1, whereas all of the healthy individuals have test results of a minimum number of swallows of ≤1.

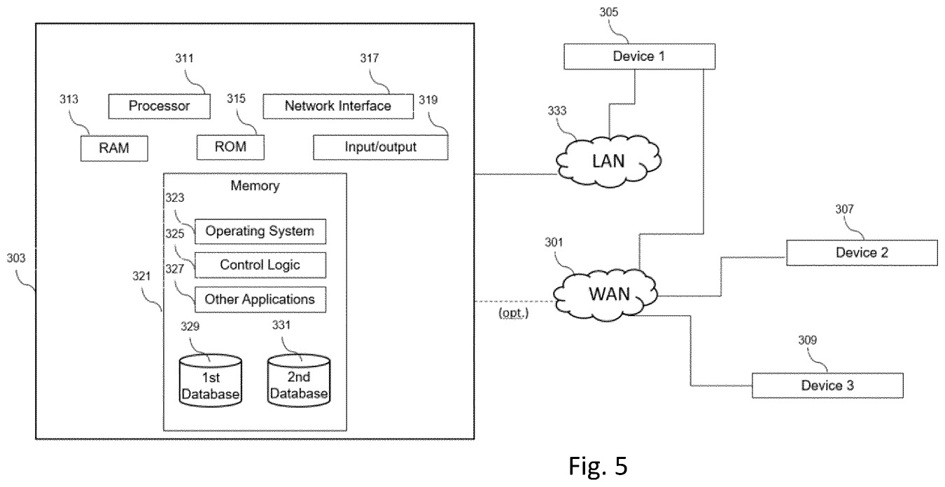

FIG. 5 illustrates an example of a network architecture and data processing device that may be used to implement one or more illustrative aspects described herein, such as the aspects described in FIGS. 1 and 2. Various network nodes 303, 305, 307, and 309 may be interconnected via a wide area network (WAN) 301, such as the Internet. Other networks may also or alternatively be used, including private intranets, corporate networks, LANs, wireless networks, personal networks (PAN), and the like. Network 301 is for illustration purposes and may be replaced with fewer or additional computer networks. A local area network (LAN) may have one or more of any known LAN topology and may use one or more of a variety of different protocols, such as Ethernet. Devices 303, 305, 307, 309 and other devices (not shown) may be connected to one or more of the networks via twisted pair wires, coaxial cable, fibre optics, radio waves or other communication media.

The term “network” as used herein and depicted in the drawings refers not only to systems in which remote storage devices are coupled together via one or more communication paths, but also to stand-alone devices that may be coupled, from time to time, to such systems that have storage capability. Consequently, the term “network” includes not only a “physical network” but also a “content network,” which is comprised of the data-attributable to a single entity which resides across all physical networks.

The components may include data server 303, web server 305, and client computers 307, 309. Data server 303 provides overall access, control and administration of databases and control software for performing one or more illustrative aspects described herein. Data server 303 may be connected to web server 305 through which users interact with and obtain data as requested. Alternatively, data server 303 may act as a web server itself and be directly connected to the Internet. Data server 303 may be connected to web server 305 through the network 301 (e.g., the Internet), via direct or indirect connection, or via some other network. Users may interact with the data server 303 using remote computers 307, 309, e. g., using a web browser to connect to the data server 303 via one or more externally exposed web sites hosted by web server 305. Client computers 307, 309 may be used in concert with data server 303 to access data stored therein, or may be used for other purposes. For example, from client device 307 a user may access web server 305 using an Internet browser, as is known in the art, or by executing a software application that communicates with web server 305 and/or data server 303 over a computer network (such as the Internet). In some cases, the client computer 307 may be a smartphone, smartwatch or other mobile computing device, and may implement a diagnostic device, such as the device 105 shown in FIG. 1. In some cases, the data server 303 may implement a server, such as the server 150 shown in FIG. 1.

Servers and applications may be combined on the same physical machines, and retain separate virtual or logical addresses, or may reside on separate physical machines. FIG. 1 illustrates just one example of a network architecture that may be used, and those of skill in the art will appreciate that the specific network architecture and data processing devices used may vary, and are secondary to the functionality that they provide, as further described herein. For example, services provided by web server 305 and data server 303 may be combined on a single server.

Each component 303, 305, 307, 309 may be any type of known computer, server, or data processing device. Data server 303, e.g., may include a processor 311 controlling overall operation of the rate server 303. Data server 303 may further include RAM 313, ROM 315, network interface 317, input/output interfaces 319 (e.g., keyboard, mouse, display, printer, etc.) , and memory 321. I/O 319 may include a variety of interface units and drives for reading, writing, displaying, and/or printing data or files. Memory 321 may further store operating system software 323 for controlling overall operation of the data processing device 303, control logic 325 for instructing data server 303 to perform aspects described herein, and other application software 327 providing secondary, support, and/or other functionality which may or may not be used in conjunction with other aspects described herein. The control logic may also be referred to herein as the data server software 325. Functionality of the data server software may refer to operations or decisions made automatically based on rules coded into the control logic, made manually by a user providing input into the system, and/or a combination of automatic processing based on user input (e.g., queries, data updates, etc.).

Memory 321 may also store data used in performance of one or more aspects described herein, including a first database 329 and a second database 331. In some cases, the first database may include the second database (e.g., as a separate table, report, etc.). That is, the information can be stored in a single database, or separated into different logical, virtual, or physical databases, depending on system design. Devices 305, 307, 309 may have similar or different architecture as described with respect to device 303. Those of skill in the art will appreciate that the functionality of data processing device 303 (or device 305, 307, 309) as described herein may be spread across multiple data processing devices, for example, to distribute processing load across multiple computers, to segregate transactions based on geographic location, user access level, quality of service (Qos), etc.

One or more aspects described herein may be embodied in computer-usable or readable data and/or computer-executable instructions, such as in one or more program modules, executed by one or more computers or other devices as described herein. Generally, program modules include routines, programs, objects, components, data structures, etc. that perform particular tasks or implement particular abstract data types when executed by a processor in a computer or other device.

The modules may be written in a source code programming language that is subsequently compiled for execution, or may be written in a scripting language such as (but not limited to) HTML or XML. The computer executable instructions may be stored on a computer-readable medium such as a hard disk, optical disk, removable storage media, solid state memory, RAM, etc. As will be appreciated by one of skill in the art, the functionality of the program modules may be combined or distributed as desired in various embodiments. In addition, the functionality may be embodied in whole or in part in firmware or hardware equivalents such as integrated circuits, field programmable gate arrays (FPGA), and the like. Particular data structures may be used to more effectively implement one or more aspects, and such data structures are contemplated within the scope of computer executable instructions and computer-usable data described herein.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

1. A diagnostic device configured to count a number of swallows of a user, the device comprising:
at least one processor;
a user interface;
one or more sensors associated with the device; and
a memory storing computer-readable instructions that, when executed by the at least one processor, cause the diagnostic device to conduct a swallow test, the swallow test causing the diagnostic device to:
prompt, via the user interface, the user to provide a user input via the one or more sensors associated with the device each time the user is at a predetermined point during a swallowing action;
receive a plurality of inputs via the one or more sensors, each user input corresponding to a respective time at which the user is at a predetermined point during the swallowing action;
generate, in response to receiving each user input, a timestamp associated with the respective user input;
apply a counting model to data comprising the plurality of generated timestamps, wherein the counting model is configured to calculate a number of swallows of a user by counting the total number of timestamps in the data comprising the plurality of generated timestamps; and
outputting the calculated number of swallows.

2. A diagnostic device according to claim 1, wherein:
the diagnostic device comprises a smartphone comprising a display component configured to display the user interface.

3. A diagnostic device according to claim 2 wherein:
the display component comprises a touch screen comprising the one or more sensors, and wherein the user input is a screen touch detectable by the one or more sensors.

4. A diagnostic device according to claim 3, wherein the one or more sensors comprise capacitive sensors.

5. A diagnostic device according to claim 1, wherein:
the computer-readable instructions, when executed by the at least one processor, further cause the diagnostic device to prompt the user, via the user interface, to consume a piece of food.

6. A diagnostic device according to claim 1, wherein:
the computer-readable instructions, when executed by the at least one processor, further cause the diagnostic device to prompt the user, via the user interface, to consume a drink.

7. A diagnostic device according to claim 1, wherein:
the predetermined point during a swallowing action is a beginning or end of the swallowing action.

8. A diagnostic device according to claim 1, wherein the computer-readable instructions, when executed by the at least one processor, causes the diagnostic device to conduct a plurality of swallow tests.

9. A diagnostic device according to claim 8 wherein the computer-readable instructions, when executed by the at least one processor, causes the diagnostic device to apply a clinical interpretation model to a plurality of the calculated numbers of swallows, wherein the clinical interpretation model outputs an indication of the presence or absence of a muscular disability.

10. A diagnostic device according to claim 9, wherein the clinical interpretation model is configured to determine a minimum number of swallows in the plurality of the calculated numbers of swallows.

11. A diagnostic device according to claim 10, wherein the clinical interpretation model is configured to compare the minimum number of swallows to a predetermined value and, based on this comparison, to output the indication of the presence or absence of the muscular disability.

12. A diagnostic device according to claim 11, wherein the clinical interpretation model is configured to determine whether the minimum number of swallows is greater than a predetermined threshold, and, if it is determined that the minimum number of swallows is greater than the predetermined threshold, to output an indication of the presence of the muscular disability, and if it is determined that the minimum number of swallows is less than or equal to the predetermined threshold, to output an indication of the absence of the muscular disability.

13. A diagnostic device according to claim 12, wherein the predetermined threshold is 1.

14. A computer-implemented method of counting a number of swallows of a subject, the computer-implemented method comprising:
prompting, via a user interface, the subject to provide a user input via one or more sensors associated with a diagnostic device each time the user is at a predetermined point during a swallowing action;
receiving a plurality of inputs via the one or more sensors, each user input corresponding to a respective time at which the user is at a predetermined point during the swallowing action;
generating, in response to receiving each user input, a timestamp associated with the respective user input;
applying a counting model to data comprising the plurality of generated timestamps, wherein the counting model is configured to calculate a number of swallows of a user by counting the total number of timestamps in the data comprising the plurality of generated timestamps.

15. A computer-implemented method according to claim 14, wherein the computer-implemented method further comprises the steps of:
applying a clinical interpretation model to the calculated number of swallows, wherein the clinical interpretation model outputs an indication of the presence or absence of a muscular disability, or an indication of the progression of a muscular disability.

16. (canceled)

17. A computer-implemented method according to claim 14, wherein the steps of prompting the subject, receiving the user inputs and generating the timestamps are carried out by a processor of a diagnostic device, and wherein the step of applying the counting model is carried out by a processor of a server, wherein the diagnostic device is configured to transmit the generated time stamps to the server, and wherein the diagnostic device comprises:
at least one processor;
a user interface;
one or more sensors associated with the device; and
a memory storing computer-readable instructions that, when executed by the at least one processor, cause the diagnostic device to conduct a swallowing test, the swallowing test causing the diagnostic device to:
prompt, via the user interface, the user to provide a user input via the one or more sensors associated with the device each time the user is at a predetermined point during a swallowing action;
receive a plurality of user inputs via the one or more sensors, each user input corresponding to a respective time at which the user is at a predetermined point during a swallowing action;
generate, in response to receiving each user input, a timestamp associated with the respective user input.
